(19) United States
(12) Patent Application Publication
(10) Pub. No.: US 2015/0157207 A1
(43) Pub. Date: Jun. 11, 2015

(54) SCAN PROTOCOL ADJUSTING APPARATUS, IMAGING APPARATUS, AND METHOD FOR ADJUSTING SCAN PROTOCOL

(71) Applicant: GE Medical System Global Technology Company, LLC, Waukesha, WI (US)

(72) Inventors: Hitoshi Ikeda, Tokyo (JP); Yoshihiro Tomoda, Tokyo (JP); Marie Uno, Tokyo (JP)

(21) Appl. No.: 14/411,054

(22) PCT Filed: May 31, 2013
(86) PCT No.: PCT/US2013/043591

§ 371 Date: Dec. 23, 2014

(30) Foreign Application Priority Data
Jun. 27, 2012 (JP) 2012-144417

(51) Int. Cl. A61B 5/00; A61B 5/055

(52) U.S. Cl. CPC A61B 5/0037; A61B 5/055; A61B 5/7221

(57) ABSTRACT

A scan protocol adjusting apparatus includes a setting device which sets a scan protocol of a plurality of scans included in one examination, a designating device which designates a target value of examination time, and an adjusting device which adjusts the set scan protocol so that a prediction value of the examination time of the one examination becomes close to the target value of the examination time.

### TECHNICAL FIELD

The present invention relates to a scan protocol adjusting apparatus for adjusting a scan protocol, an imaging apparatus, and a method for adjusting a scan protocol.

### BACKGROUND ART

In a facility such as hospital and medical examination center, an examination using an imaging apparatus such as a magnetic resonance imaging apparatus or a radiation tomographic apparatus is conducted on a number of subjects every day. Examination time per subject is determined in advance and, on the basis of the time, a schedule of examinations on a number of subjects is made. Usually, a plurality of scan protocols are prepared and stored in an imaging apparatus so that a necessary kind of image can be captured smoothly at desired image quality within the determined examination time, and some optimum scan protocols are selected and used in accordance with a region to be examined and a purpose.

On the other hand, in reality, it is rare that the examination which is planned as described above is conducted as scheduled and, in many cases, the examination time has to be shortened at a certain timing. For example, in the case where an urgent examination has to be conducted or in the case where time for a preceding examination becomes longer than scheduled time, for the next subject, an examination has to be finished in time shorter than the predetermined time.

In such a case, the operator has to adjust the scan protocol with some compromise so that the examination is completed within predetermined time shorter than the determined time. The scan protocol is made of a number of parameters each of which is finely sophisticatedly set. The operator usually wishes to maintain image contrast although scan time is shortened in each of scans. Consequently, in each of scans, the operator has to adjust the parameters of each scan so that scan time is shortened and entire examination time is within predetermined time while maintaining the image contrast. However, to perform such adjustment smoothly, experience and knowledge to certain degree is necessary. In addition, since the adjusting work itself is very complicated, the burden on the operator is heavy.

As means solving the problem, for example, as proposed in patent literature 1 and the like, an imaging apparatus is provided with a function of automatically adjusting parameters of a scan so that the scan time becomes shortest, and the operator uses the function to shorten the examination time.

[Patent Literature 1] Japanese Unexamined Patent Application Publication No. 2011-229546

### TECHNICAL PROBLEM

However, generally, scan time and the quality of an image obtained by the scan such as image SNR (signal noise ratio) or spatial resolution have a trade-off relation. Consequently, when the parameters of the scan are adjusted so that the scan time becomes the shortest, given examination time cannot be maximally utilized, and the quality of the image is reduced more than necessary.

For such reasons, a technique capable of easily setting a scan protocol which suppresses deterioration in the quality of an image obtained as much as possible while completing a series of scans in given examination time is demanded.

### SOLUTION TO PROBLEM

The invention according to a first aspect provides a scan protocol adjusting apparatus including: a setting device which sets a scan protocol of a plurality of scans included in one examination; a designating device which designates a target value of examination time; and an adjusting device which adjusts the set scan protocol so that a prediction value of the examination time of the one examination becomes close to the target value of the examination time.

The invention of a second aspect provides the scan protocol adjusting apparatus of the first aspect in which priority is set for the scan protocols of the plurality of scans, and the adjusting device adjusts the scan protocol in accordance with the priority.

The invention of a third aspect provides the scan protocol adjusting apparatus of the first aspect, in which the adjusting device adjusts the set scan protocol so that scan time of each of the plurality of scans is changed at a substantially equal ratio.

The invention of a fourth aspect provides the scan protocol adjusting apparatus according to any one of the first to third aspects, in which the prediction value of the examination time includes time required to execute a prescan which is executed before each of the plurality of scans.

The invention of a fifth aspect provides the scan protocol adjusting apparatus according to any one of the first to fourth aspects, in which the prediction value of the examination time includes time between scans in the plurality of scans.

The invention of a sixth aspect provides the scan protocol adjusting apparatus according to any one of the first to fifth aspects, further including selecting device which selects a desired image quality item of an image obtained by the plurality of scans, and the adjusting device adjusts a parameter so as to maintain image quality of the selected image quality item.

The invention of a seventh aspect provides the scan protocol adjusting apparatus of the sixth aspect, in which the selecting device selects image SNR or spatial resolution as the image quality item.

The invention of an eighth aspect provides an imaging apparatus including the scan protocol adjusting apparatus according to any one of the first to seventh aspects.

The invention of a ninth aspect provides the imaging apparatus of the eighth aspect which performs magnetic resonance imaging.

The invention of a tenth aspect provides a program for making a computer function as the scan protocol adjusting apparatus according to any one of the first to seventh aspects.

### ADVANTAGEOUS EFFECTS OF INVENTION

According to the invention of the above-described aspects, when the operator designates a target value of examination time, a set scan protocol is adjusted so that a prediction value of examination time of one examination including a plurality of scans becomes close to the designated target value of the examination time. Consequently, a series of scans are performed in given examination time and the scan protocol by which the given examination time is effectively maximally used can be automated. While completing the series of scans in the given examination time, the scan protocol which can suppress deterioration in the quality of an image obtained can be easily set.

Further objects and advantages of the embodiments described herein will be apparent from the following description as illustrated in the accompanying drawings.

### BRIEF DESCRIPTION OF DRAWINGS

FIG. 1 is a schematic diagram of a magnetic resonance imaging apparatus according to an embodiment of the invention.

FIG. 2 is a diagram illustrating a flow of making a scan plan.

FIG. 3 is a flowchart of protocol adjustment by a first method.

FIG. 4 is a diagram illustrating an example of the protocol adjustment by the first method.

FIG. 5 is a flowchart of protocol adjustment by a second method.

FIG. 6 is a diagram illustrating an example of the protocol adjustment by the second method.

### DESCRIPTION OF EMBODIMENTS

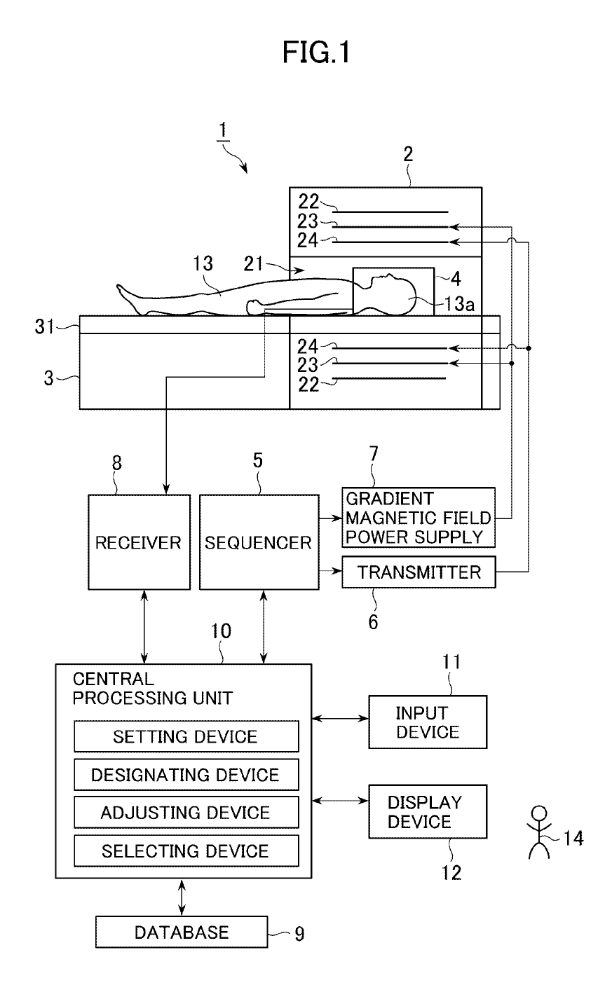

FIG. 1 is a schematic diagram of a magnetic resonance imaging apparatus according to an embodiment.

A magnetic resonance imaging apparatus (hereinbelow, called MRI apparatus) 1 includes a magnetic field generating unit 2, a table 3, and a reception coil 4.

The magnetic field generating unit 2 has a bore 21 in which a subject 13 is housed, a superconducting coil 22, a gradient coil 23, and a transmission coil 24. The superconducting coil 22 forms a magnetostatic field B0, the gradient coil 23 forms a gradient magnetic field, and the transmission coil 24 transmits an RF (Radio Frequency) pulse. The subject 13 is an example of an object to be scanned in the invention.

The table 3 has a cradle 31 for carrying the subject 13. By the cradle 31, the subject 13 is carried into the bore 21.

The reception coil 4 is attached to, for example, the head 13a of the subject 13 and receives a magnetic resonance signal from the head 13a.

The MRI apparatus 1 also includes a sequencer 5, a transmitter 6, a gradient magnetic field power supply 7, a receiver 8, a database 9, a central processing unit 10, an input device 11, and a display device 12.

The sequencer 5 transmits information of the RF pulse (center frequency, band width, and the like) to the transmitter 6 and transmits information of the gradient magnetic field (intensity of the gradient magnetic field and the like) to the gradient magnetic field power supply 7 under control of the central processing unit 10.

The transmitter 6 drives the transmission coil 24 on the basis of the information transmitted from the sequencer 5.

The gradient magnetic field power supply 7 drives the gradient coil 23 on the basis of the information transmitted from the sequencer 5.

The receiver 8 processes the magnetic resonance signal received by the reception coil 4 and transmits the resultant signal to the central processing unit 10.

The database 9 stores data of a reconstructed image, a scan protocol, a program, and the like.

The central processing unit 10 generally controls the operations of the units in the MRI apparatus 1 so as to realize various operations of the MRI apparatus 1 by, for example, reconstructing an image on the basis of a signal received from the receiver 8. On the basis of information entered by an operator 14 with the input device 11, the central processing unit 10 selects a protocol of each of a plurality of scans (scan protocol) necessary for an examination from protocols which are prestored in the database 9 and sets it. Further, the central processing unit 10 adjusts the set protocol so that a prediction value of examination time becomes close to a given target value of examination time. The central processing unit 10 is constructed by, for example, a computer. The central processing unit 10 performs processing s of a setting device, a designating device, an adjusting device and a selecting device in the invention by executing a predetermined program.

The input device 11 enters various instructions to the central processing unit 10 in accordance with an operation of the operator 14. The display device 12 displays various information.

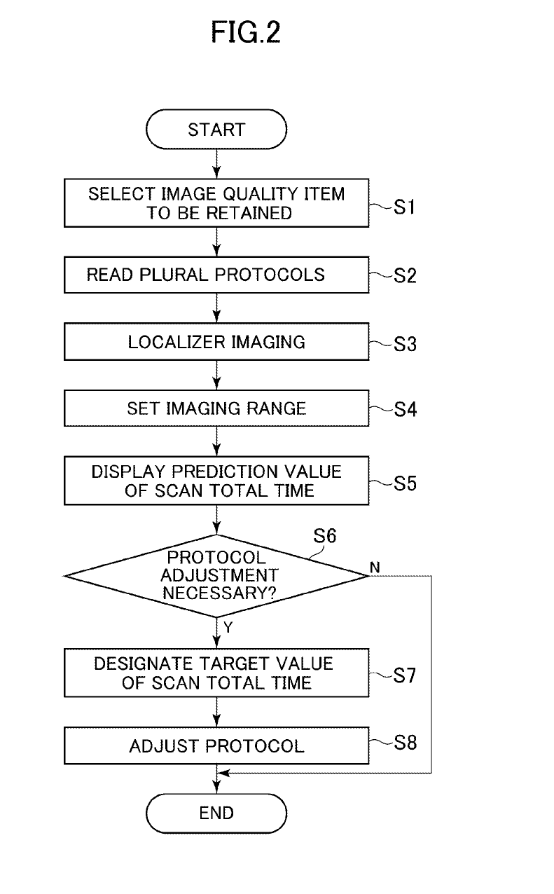

FIG. 2 is a diagram illustrating a flow when the operator 14 makes a scan plan.

In step S1, the operator preliminarily selects an image quality item desired to be retained (hereinbelow, called retention image quality item) at the time of protocol adjustment. In this case, options of the retention image quality items are set as image SNR and spatial resolution. The operator selects any of the options.

In step S2, the operator selects protocols of a plurality of scans necessary for an examination on the basis of information such as the physical constitution of the subject, the object of the examination, and a region to be imaged. Examples of the plurality of scans include a scan for obtaining a T1-weighted image, a scan for obtaining a T2-weighted image, and a scan for obtaining a FLAIR (FLuid-Attenuated Inversion Recovery) image.

In step S3, the operator executes localizer imaging on the subject by a predetermined operation.

In step S4, the operator refers to an image obtained by the localizer imaging and sets a part of a protocol, for example, parameters such as an imaging range and FOV (Field Of View).

In step S5, the MRI apparatus 1 calculates a prediction value of scan total time and displays it on the basis of the set protocol.

In step S6, the operator refers to the prediction value of the scan total time and determines whether protocol adjustment for shortening the examination time is performed or not. In the case of performing the protocol adjustment, the program advances to step S7. In the case where the protocol adjustment is not performed, the scan plan is finished.

In step S7, the operator designates a target value of the examination time. For example, when the examination time has to be shortened from planned 20 minutes to 15 minutes, the target value of the examination time is designated as 15 minutes.

In step S8, the protocol of at least one of a plurality of scans to be executed is adjusted so that the prediction value of the examination time becomes close to the target value.

A specific method of the protocol adjustment in step S8 will be described together with its example.

First Method

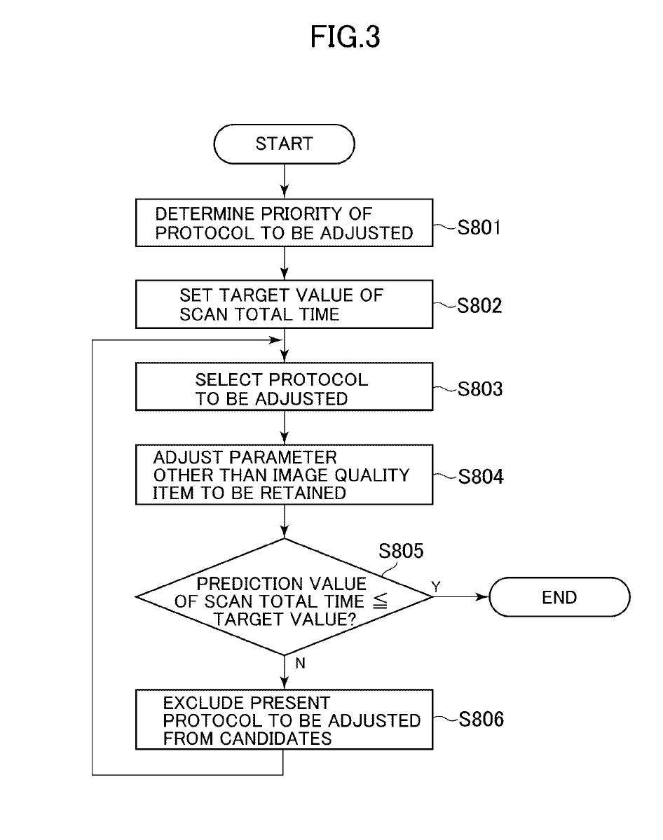

FIG. 3 is a flowchart of protocol adjustment by a first method.

In step S801, priority at the time of adjusting a parameter for the protocol of each of the scans to be executed is determined. The priority may be determined arbitrarily by the operator or determined according to the order of the scans to be executed, the order opposite to the order of the scans to be executed, the order of importance of the scans, or the like.

In step S802, time obtained by subtracting total time of a prescan from the designated target value of the examination time is set as a target value of scan total time. The prescan denotes calibration of a setting executed so that imaging is performed correctly prior to start of a scan. In the prescan, for example, tuning of a coil, setting of the center frequency, adjustment of a transmission power attenuator, adjustment of reception sensitivity, and the like are performed.

In step S803, a protocol to be adjusted is selected according to the priority.

In step S804, in a protocol to be adjusted, a parameter is adjusted within an acceptable range while maintaining the image quality determined in the retention image quality item so that the prediction value of the scan total time becomes close to the target value of the scan total time. For example, when the retention image quality item is spatial resolution, the number of matrixes in the phase encoding direction, a parameter determining the spatial resolution, for example, the number of matrixes in the frequency encoding direction (frequency), FOV (Field Of View), slice thickness, and the like are not adjusted. The other parameters such as the number of addition times (NEX) and the band width (BW) are adjusted within the acceptable range. When the retention image quality item is an image SNR, parameters determining the image SNR, such as the number of addition times (NEX), band width (BW), the number of matrixes in the phase encoding direction (phase), the number of matrixes in the frequency encoding direction (frequency), FOV (Field Of View), and slice thickness are not adjusted, but the other parameters are adjusted within the acceptable range. A plurality of parameters is adjusted, for example, according to predetermined priority. The acceptable range of each parameter is set, for example, on condition that an image at a level which can be provided for image diagnosis can be obtained. In the prediction value of the scan total time, an interval time between scans to be executed may be predicted and included.

In step S805, whether the relation “prediction value TSp of the scan total time≦target value TSt of the scan total time” is satisfied or not is determined. In the case of YES, the protocol adjustment is finished. In the case of NO, the program advances to step S806.

In step S806, a protocol to be adjusted at the present time point is excluded from candidates of protocols to be adjusted. The program returns to step S803 and continues the process.

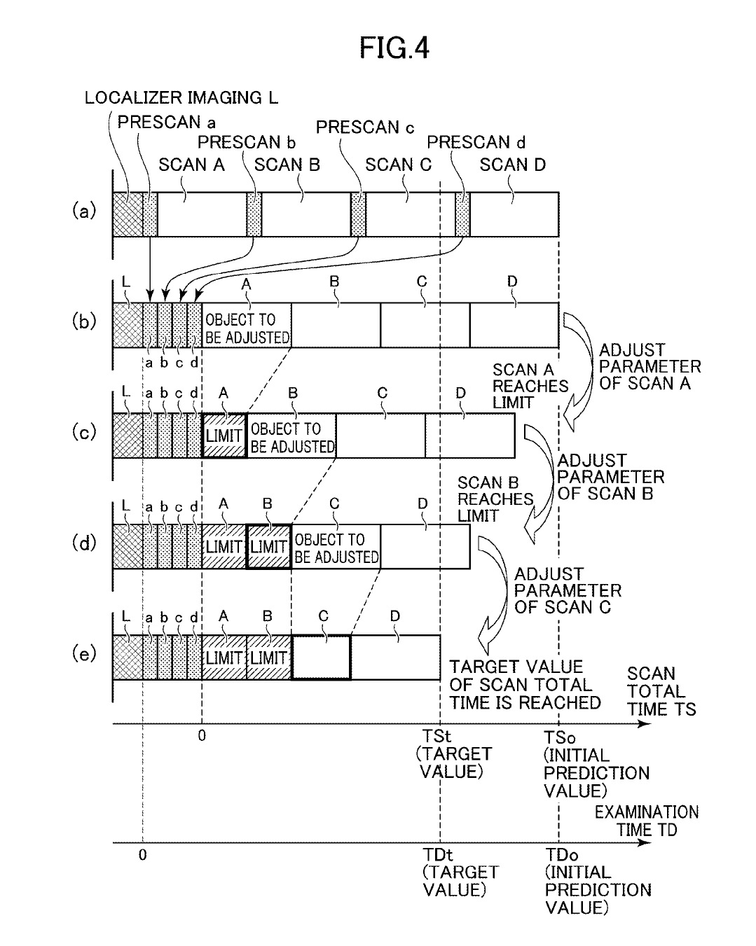

An example of the protocol adjustment by the first method will now be described. FIG. 4 is a diagram illustrating an example of the protocol adjustment by the first method and illustrating examination time including time of each of scans to be executed in the form of a graph.

In the example, as illustrated in FIG. 4(a), protocols are set so that scans A to D are executed in this order after localizer imaging L. Prescans “a” to “d” are executed before the scans A to D, respectively. In FIGS. 4(b) to 4(e), the positions of the prescans are rearranged so that the scan total time can be easily seen visually, and actual execution order is not shown.

In step S801, the priority of protocols to be adjusted is set to the order of protocols of the scans A to D which is the same order of scans to be executed.

In step S802, as illustrated by the horizontal axis of the graph of FIG. 4, the total time of the prescans “a” to “d” is subtracted from the target value TDt of the examination time to obtain the target value TSt of the scan total time.

In step S803, as illustrated in FIG. 4(b), first, the protocol of the scan A is set as an object to be adjusted.

In step S804, as illustrated in FIG. 4(c), the scan time is shortened in the protocol of the scan A and the parameter is adjusted within the acceptable range so that the prediction value TSp of the scan total time becomes close to the target value TSt. When the parameter reaches the border of the acceptable range, the adjustment is temporarily stopped. In the example of FIG. 4(c), the parameter reaches the border of the acceptable range, the scan time of the scan A reaches the limit (shortest), and the adjustment is temporarily stopped.

In step S805, as illustrated in FIG. 4(c), the relation “prediction value TSp of the scan total time≦target value TSt of scan total time” is not satisfied. The program advances to step S806.

In step S806, the protocol of the scan A as the object to be adjusted at present is excluded from candidates of objects to be adjusted. The program returns to step S803.

In step S803 of the second time, as illustrated in FIG. 4(c), the protocol of the scan B having the second highest priority is selected as an object to be adjusted.

In step S804 of the second time, the parameter adjustment is performed as illustrated in FIG. 4(d). In the example of FIG. 4(d), the parameter reaches the border of the acceptable range, the scan time of the scan B reaches the limit (shortest), and the adjustment is temporarily stopped.

In step S805 of the second time, the relation “the prediction value TSp of the scan total time≦target value TSt of the scan total time” is not satisfied as illustrated in FIG. 4(d). The program advances to step S806.

In step S806 of the second time, the protocol of the scan B is excluded from candidates of objects to be adjusted.

In step S803 of the third time, the protocol of the scan C having the third highest priority is selected as an object to be adjusted as illustrated in FIG. 4(d).

In step S804 of the third time, the parameter adjustment is performed as illustrated in FIG. 4(e). In the example of FIG. 4(e), before the parameter reaches the border of the acceptable range, the relation “prediction value TSp of the scan total time≦target value TSt of the scan total time” is satisfied.

In step S805 of the third time, since the relation “prediction value TSp of scan total time≦target value TSt of scan total time” is satisfied, the protocol adjustment is finished.

Second Method

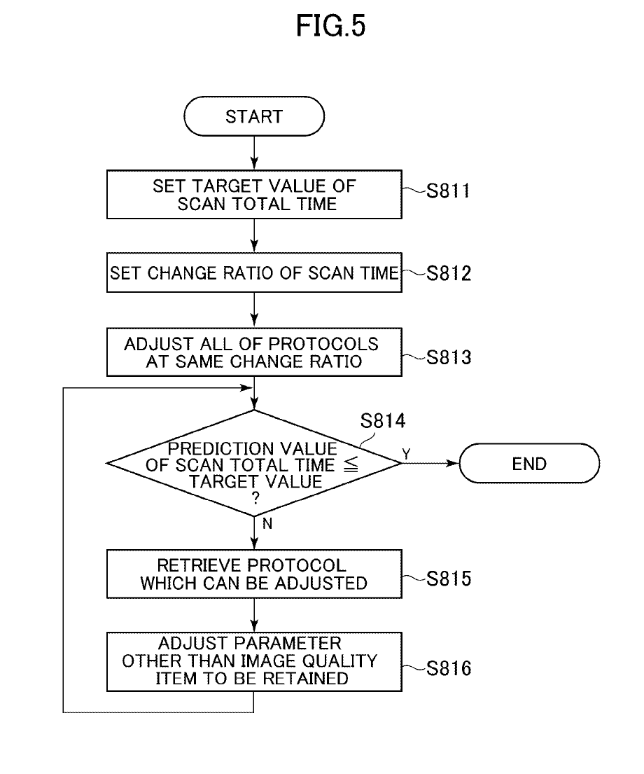

FIG. 5 is a flowchart of protocol adjustment by a second method.

In S811, time obtained by subtracting the total time of the prescan from the target value of the examination time designated is set as the target value of the scan total time.

In S812, “target value of scan total time/initial prediction value of scan total time” is set as the ratio to be changed with respect to the time of each of the scans. The initial prediction value of the scan total time is a prediction value of the scan total time obtained on the basis of the protocol immediately after the setting, that is, before the adjustment.

In S813, the parameter is adjusted within the acceptable range while maintaining the image quality of the retention image quality item selected in the protocol of each scan so that the time of each scan to be executed becomes the ratio which is set in step S812.

In S814, whether the relation “prediction value of scan total time≦target value of scan total time” is satisfied or not is determined. In the case of YES, the protocol adjustment is finished. In the case of NO, the program advances to S815.

In S815, a protocol whose parameter is within the acceptable range and, further, which can be adjusted is retrieved, and the retrieved protocol is selected as an object to be adjusted.

In S816, the parameter is adjusted within the acceptable range while maintaining the image quality of the retention image quality item selected so that the prediction value of the scan total time becomes close to the target value of the scan total time in the protocol as an object to be adjusted. After that, the program returns to S814.

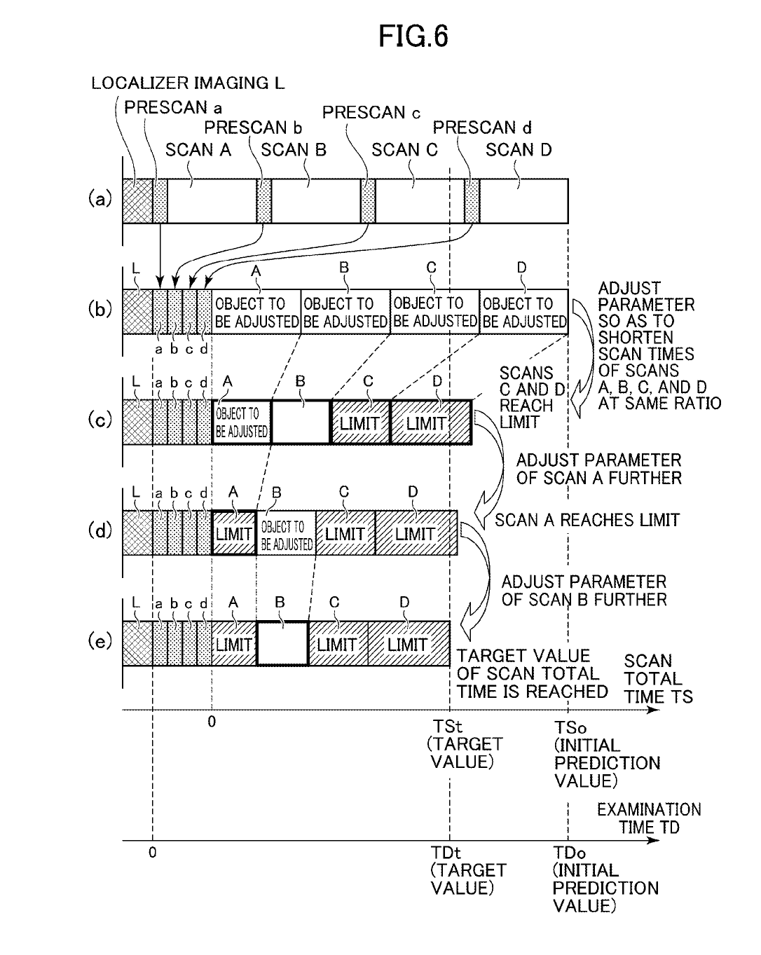

An example of the protocol adjustment by the second method will now be described. FIG. 6 is a diagram illustrating an example of the protocol adjustment by the second method. Like FIG. 4, the examination time including the scan time of each scan to be executed is expressed as a graph.

In the example, as illustrated in FIG. 6(a), like the example of FIG. 4, protocols are set so that the scans A to D are executed in this order after the localizer imaging L. The prescans “a” to “d” are executed before the scans A to D, respectively.

In step S811, as illustrated by the horizontal axis of the graph of FIG. 6, the total time of the prescans “a” to “d” is subtracted from the target value TDt of the examination time to obtain the target value TSt of the scan total time.

In step S812, a change ratio W of the scan times of the scans A to D is set. The change ratio W is obtained as “target value TSt of scan total time/initial prediction value TSo of scan total time).

In step S813, the parameter of each protocol is adjusted within the acceptable range so that the scan time of each of the scans A to D becomes time obtained by multiplying the scan time with the change ratio W. For example, when it is assumed that the change range W is 0.7, the adjustment is performed so that the scan time of each of the scans A to D becomes 70% of scan time predicted by the initial protocol. In the example of FIG. 6(c), as a result of changing the scan time of each of the scans A to D by the same change ratio W, the scan time can be shortened to the target scan time in the protocols of the scans A and B. In the protocols of the scans C and D, the parameters reach the border of the acceptable range, and the scan time of the scans C and D reaches the limit (shortest).

In step S814, as illustrated in FIG. 6(c), the relation “prediction value TSp of the scan total time≦target value TSt of the scan total time” is not satisfied. The program advances to step S815.

In step S815, a protocol whose parameter is within the acceptable range and which can be adjusted is retrieved. As a result, the protocol of the scan A is retrieved. As illustrated in FIG. 6(c), the protocol of the scan A is set as the next object to be adjusted.

In step S816, as illustrated in FIG. 6(d), the parameter of the protocol of the scan A is adjusted. In the example of FIG. 6(d), the parameter reaches the border of the acceptable range, the scan time of the scan A also reaches the border (shortest), and the adjustment is temporarily stopped. The program returns to step S814.

In step S814 of the second time, as illustrated in FIG. 6(d), the relation “prediction value TSp of the scan total time≦target value TSt of the scan total time” is not satisfied. The program advances to step S815.

In step S815 of the second time, the protocol whose parameter is within the acceptable range and which can be adjusted is retrieved. The protocol of the scan B is retrieved. As illustrated in FIG. 6(d), the protocol of the scan B is set as the next object to be adjusted.

In step S816 of the second time, as illustrated in FIG. 6(e), the parameter of the protocol of the scan B is adjusted. In the example of FIG. 6(e), before the parameter reaches the border of the acceptable range, the prediction value TS of the scan total time reaches the target value TSt. The program returns to step S814.

In step S814 of the third time, as illustrated in FIG. 6(e), “prediction value TSp of scan total time≦target value TSt of scan total time” is satisfied. Consequently, the protocol adjustment is finished.

According to the embodiment as described above, when the operator designates a target value of examination time, a scan protocol which is set is adjusted so that a prediction value of examination time of an examination including a plurality of scans becomes close to the designated target value of the examination time. Therefore, a series of scans are performed in given examination time and the scan protocol by which the given examination time is effectively maximally used can be automated. While completing the series of scans in the given examination time, the scan protocol which can suppress deterioration in the quality of an image obtained can be easily set.

Particularly, in the embodiment, the examination time can be optimized in an entire examination including a plurality of scans, not in a scan unit, so that a more efficient and well-planned examination can be expected.

Embodiments of the invention are not limited to the foregoing embodiment, and the invention can be variously changed without departing from the gist of the invention.

For example, an object to be examined is not limited to a human being but may be another animal.

A scan protocol adjusting device of adjusting a protocol of a scan as in the foregoing embodiment and a program for making a computer function as the device are also embodiments of the invention.

Although a magnetic resonance imaging apparatus has been described in the foregoing embodiment, the invention can be also applied to other imaging apparatuses such as an X-ray CT (Computed Tomography) apparatus.

Many wide different embodiments of the invention may be configured without departing from the spirit and the scope of the present invention. It should be understood that the present invention is not limited to the specific embodiments described in the specification, except as defined in the appended claims.

### INDUSTRIAL APPLICABILITY

The present invention is applied to the apparatus which sets a scan protocol, and the apparatus can shorten examination time, suppressing deterioration in the quality of an image the present invention.

1. A scan protocol adjusting apparatus, comprising:
a setting device configured to set a scan protocol of a plurality of scans included in one examination;
a designating device configured to designate a target value of examination time; and
an adjusting device configured to adjust the set scan protocol so that a prediction value of the examination time of the one examination becomes close to the target value of the examination time.

2. The scan protocol adjusting apparatus according to claim 1, wherein priority is set for the scan protocols of the plurality of scans, and the adjusting device adjusts the scan protocol in accordance with the priority.

3. The scan protocol adjusting apparatus according to claim 1, wherein the adjusting device adjusts the set scan protocol so that scan time of each of the plurality of scans is changed at a substantially equal ratio.

4. The scan protocol adjusting apparatus according to claim 1, wherein the prediction value of the examination time includes time required to execute a prescan which is executed before each of the plurality of scans.

5. The scan protocol adjusting apparatus according to claim 2, wherein the prediction value of the examination time includes time required to execute a prescan which is executed before each of the plurality of scans.

6. The scan protocol adjusting apparatus according to claim 3, wherein the prediction value of the examination time includes time required to execute a prescan which is executed before each of the plurality of scans.

7. The scan protocol adjusting apparatus according to claim 1, wherein the prediction value of the examination time includes time between scans in the plurality of scans.

8. The scan protocol adjusting apparatus according to claim 2, wherein the prediction value of the examination time includes time between scans in the plurality of scans.

9. The scan protocol adjusting apparatus according to claim 3, wherein the prediction value of the examination time includes time between scans in the plurality of scans.

10. The scan protocol adjusting apparatus according to claim 4, wherein the prediction value of the examination time includes time between scans in the plurality of scans.

11. The scan protocol adjusting apparatus according to claim 5, wherein the prediction value of the examination time includes time between scans in the plurality of scans.

12. The scan protocol adjusting apparatus according to claim 1, further comprising:
a selecting device configured to select a desired image quality item of an image obtained by the plurality of scans,
wherein the adjusting device adjusts a parameter so as to maintain quality of the selected image quality item.

13. The scan protocol adjusting apparatus according to claim 2, further comprising:
a selecting device configured to select a desired image quality item of an image obtained by the plurality of scans,
wherein the adjusting device adjusts a parameter so as to maintain quality of the selected image quality item.

14. The scan protocol adjusting apparatus according to claim 3, further comprising:
a selecting device configured to select a desired image quality item of an image obtained by the plurality of scans,
wherein the adjusting device adjusts a parameter so as to maintain quality of the selected image quality item.

15. The scan protocol adjusting apparatus according to claim 4, further comprising:
a selecting device configured to select a desired image quality item of an image obtained by the plurality of scans,
wherein the adjusting device adjusts a parameter so as to maintain quality of the selected image quality item.

16. The scan protocol adjusting apparatus according to claim 7, further comprising:
a selecting device configured to select a desired image quality item of an image obtained by the plurality of scans,
wherein the adjusting device adjusts a parameter so as to maintain quality of the selected image quality item.

17. The scan protocol adjusting apparatus according to claim 12, wherein the selecting device selects image SNR or spatial resolution as the image quality item.

18. An imaging apparatus comprising the scan protocol adjusting apparatus according to claim 1.

19. The imaging apparatus according to claim 18, wherein magnetic resonance imaging is performed.

20. A method for adjusting a scan protocol, the method comprising:
setting a scan protocol of a plurality of scans included in one examination;
designating a target value of examination time; and
adjusting the set scan protocol so that a prediction value of the examination time of the one examination becomes close to the target value of the examination time.
